(19) United States
(12) Patent Application Publication
(10) Pub. No.: US 2026/0315732 A1
(43) Pub. Date: Oct. 15, 2026

(54) VIBROACOUSTIC ROPE-BINDING THERAPEUTIC SYSTEM AND METHOD OF USE

(71) Applicants: Jenna Kind, Cedar Ridge, CA (US); Valeriy Rotar, Antelope, CA (US)

(72) Inventors: Jenna Kind, Cedar Ridge, CA (US); Valeriy Rotar, Antelope, CA (US)

(21) Appl. No.: 19/465,091

(22) Filed: Jan. 30, 2026

Related U.S. Application Data
(60) Provisional application No. 63/756,352, filed on Feb. 10, 2025.

(51) Int. Cl. A61M 21/02; A61F 5/37; A61G 7/10; A61M 21/00

(52) U.S. Cl. CPC A61M 21/02; A61F 5/3715; A61G 7/1051; A61M 2021/0022; A61M 2021/0027; A61M 2209/088

(57) ABSTRACT

A vibroacoustic rope-binding therapeutic system that combines Shibari-inspired rope binding with vibroacoustic therapy is disclosed. The system includes a suspension framework supporting a plurality of ropes configured to form a rope-binding configuration with one or more tie points for supporting a user. One or more suspension ring assemblies are coupled to the framework and include an anchoring element for receiving the ropes. A vibroacoustic transducer is mechanically integrated with or coupled to the anchoring element and is configured to convert an electrical drive signal into mechanical oscillation that is transmitted through tensioned rope segments. A control unit having a signal generator, power amplifier, digital controller, and user interface generates and modulates therapeutic drive signals and energizes the transducer to deliver controlled low-frequency vibroacoustic stimulation to the user through rope-body contact regions to promote relaxation, reduce stress, and ease muscle tension.

### CROSS-REFERENCE TO RELATED APPLICATION

The present application claims priority to, and the benefit of, U.S. Provisional Application No. 63/756,352 which was filed on Feb. 10, 2025 and is incorporated herein by reference in its entirety.

### FIELD OF THE INVENTION

The present invention generally relates to the vibroacoustic treatment devices. More specifically, the present invention relates to a vibroacoustic rope-binding therapeutic system configured to combine Shibari-inspired decorative and grounding rope binding with vibroacoustic therapy to deliver controlled low-frequency vibration to a user through a rope-binding configuration. The invention comprises a multi-component therapeutic assembly including a suspension framework configured as a rigid, load-bearing structure for supporting a plurality of ropes arranged to form one or more tie points and rope-body contact regions on the user. One or more suspension ring assemblies are coupled to the suspension framework, each suspension ring assembly including an anchoring element configured to receive and retain rope segments for suspension and routing. A vibroacoustic transducer is mechanically integrated with or coupled to the anchoring element such that vibroacoustic energy generated by the transducer is transferred into the anchoring element and transmitted through tensioned rope segments to provide localized vibroacoustic stimulation. A control unit is operatively connected to the vibroacoustic transducer through a wired connection and/or wireless communication channel and includes a signal generator, a power amplifier, a digital controller, and a user interface for selecting and adjusting vibration parameters including frequency, amplitude, waveform, modulation pattern, duty cycle, and program duration. Accordingly, the present disclosure makes specific reference thereto. Nonetheless, it is to be appreciated that aspects of the present invention are also equally applicable to other like applications, devices, and methods of manufacture.

### BACKGROUND OF THE INVENTION

By way of background, Shibari is a Japanese term that translates to “binding” or “tying,” and in contemporary practice commonly refers to an aesthetic and decorative form of rope binding. Shibari is often valued for its visual artistry, tactile pressure, and perceived grounding or meditative qualities. However, traditional Shibari sessions can evoke strong emotional and physiological responses in participants. For individuals, particularly those with heightened sensitivity, anxiety, or past traumatic experiences, the intensity of the sensory stimulation and emotional activation can be overwhelming, triggering, or destabilizing without appropriate therapeutic support. Accordingly, there is a need for a solution that makes Shibari easy and convenient for users.

Vibroacoustic therapy is a separate and established wellness modality that utilizes controlled sound frequencies and low-frequency vibrations to promote relaxation, relieve stress, reduce muscle tension, and support emotional regulation. Although Shibari-inspired rope binding and vibroacoustic therapy can each produce beneficial effects independently, existing systems do not provide an integrated structure for combining these practices in a controlled therapeutic environment. Accordingly, there exists a need for a specialized system and method that safely and effectively merges rope-binding practices with vibroacoustic stimulation.

Therefore, there exists a long-felt need in the art for an improved therapeutic rope-binding system that overcomes limitations of conventional Shibari practices and conventional vibroacoustic therapy systems. There is a long-felt need for a specialized system that integrates Shibari-inspired decorative and grounding rope binding with controlled vibroacoustic stimulation in a safe, repeatable, and therapeutic manner. Additionally, there exists a need for an integrated device that delivers targeted low-frequency vibration through rope contact regions to enhance relaxation, reduce stress, and ease muscle tension while maintaining comfort and stability. Moreover, there is a need for a therapeutic system that enables customization of vibration parameters such as frequency, intensity, waveform, and modulation patterns to suit different users, sensitivities, and therapeutic objectives. Finally, there is a need for a durable, load-bearing, and user-friendly apparatus that can be deployed in clinical, wellness, or personal settings to expand access to these underutilized therapeutic practices.

The subject matter disclosed and claimed herein, in one embodiment, comprises a vibroacoustic rope-binding therapeutic system designed to provide a multi-sensory therapeutic experience by combining Shibari-inspired rope binding with vibroacoustic therapy. The system includes a suspension framework comprising a rigid, load-bearing structure supported by an overhead support surface and configured to support a rope-binding configuration for suspending or supporting a user. A plurality of ropes is provided, wherein each rope comprises an elongate flexible tensile member configured to create one or more tie points including knots, wraps, hitches, cinches, or other formations to distribute load and provide tactile grounding. The system further includes at least one suspension ring assembly coupled to the suspension framework, wherein the suspension ring assembly comprises an anchoring element configured to receive and retain one or more ropes for suspension and routing.

In one embodiment, the suspension ring assembly further comprises a vibroacoustic transducer mechanically coupled to the anchoring element. The vibroacoustic transducer is configured to convert an electrical drive signal into mechanical oscillation such that vibration is transferred into the anchoring element and into rope segments connected thereto. A control unit is provided in communication with the vibroacoustic transducer through a wired connection and/or a wireless communication channel. The control unit includes a signal generator, a power amplifier, and a digital controller configured to generate and modulate vibroacoustic drive signals in therapeutic frequency ranges and intensity levels.

In this manner, the vibroacoustic rope-binding therapeutic system of the present invention provides an apparatus and method that combines rope-binding with vibroacoustic vibration through the rope system. The invention enables controlled and customizable vibroacoustic stimulation through rope-body contact regions and maintains load-bearing suspension functionality. As a result, the system provides therapeutic effectiveness and increases accessibility of Shibari-inspired grounding practices and vibroacoustic therapy for stress reduction, muscle relaxation, and emotional regulation.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a vibroacoustic rope-binding therapeutic system. The system comprises a suspension framework configured as a rigid, load-bearing structure supported by an overhead support surface. The system further comprises a plurality of ropes configured to form a rope-binding configuration on a user, wherein each rope comprises an elongate flexible tensile member configured to form one or more tie points. At least one suspension ring assembly is coupled to the suspension framework and is configured to receive at least one rope of the plurality of ropes, and the suspension ring assembly comprises an anchoring element.

The system also comprises at least one vibroacoustic transducer mechanically coupled to the anchoring element of the suspension ring assembly, wherein the vibroacoustic transducer is configured to convert an electrical drive signal into mechanical oscillation. A control unit is provided in communication with the vibroacoustic transducer, and the control unit comprises a user interface configured to enable selection and adjustment of one or more vibration parameters. During therapeutic operation, the control unit energizes the vibroacoustic transducer to impart vibration to the anchoring element such that vibroacoustic energy is transmitted from the suspension ring assembly into the plurality of ropes and delivered to the user at one or more rope-body contact regions, thereby providing a combined therapeutic effect comprising tactile pressure stimulation from the rope-binding configuration and vibroacoustic stimulation through the plurality of ropes.

In another embodiment, the invention provides a transducer-integrated suspension ring assembly for the vibroacoustic rope-binding therapeutic system and comprises an anchoring element configured to receive and retain at least one rope used to suspend or support a user. The suspension ring assembly further comprises a vibroacoustic transducer mechanically coupled to the anchoring element, wherein the vibroacoustic transducer is configured to convert an electrical drive signal into mechanical oscillation of the anchoring element. The suspension ring assembly further comprises an electrical connection coupled to the vibroacoustic transducer and configured to receive the electrical drive signal from a control unit. In use, the mechanical oscillation of the anchoring element is transferred into the at least one rope coupled to the anchoring element. The at least one rope transmits vibroacoustic stimulation to the user at one or more rope-body contact regions.

In a further embodiment, the control unit further comprises a power amplifier configured to amplify the therapeutic vibroacoustic electrical drive signal. The control unit further comprises a digital controller configured to control operation of the signal generator and the power amplifier and to execute one or more selectable therapeutic programs. A user interface is provided and is configured to receive user input to adjust one or more vibroacoustic parameters comprising at least one of frequency, waveform shape, amplitude, pattern modulation, duty cycle, and program duration. The control unit is configured to transmit an amplified drive signal to the vibroacoustic transducer to cause vibration of an anchoring element of the suspension ring assembly. The vibration is thereby delivered through a rope system to provide vibroacoustic stimulation to a user.

In still another embodiment, a method of providing vibroacoustic therapy through a rope-binding configuration is described and comprises generating, by a control unit, a therapeutic vibroacoustic drive signal and amplifying the therapeutic vibroacoustic drive signal to produce an amplified drive signal. The method further comprises transmitting the amplified drive signal from the control unit to a vibroacoustic transducer mechanically coupled to an anchoring element of a suspension ring assembly. The method further comprises converting, by the vibroacoustic transducer, the amplified drive signal into mechanical oscillation. The method further comprises vibrating the anchoring element and one or more ropes coupled thereto such that vibration propagates along tensioned rope pathways. The method further comprises delivering vibroacoustic stimulation to a user through one or more rope-body contact regions while the user is supported by the rope-binding configuration. As a result, the user experiences a combined multi-sensory therapeutic effect comprising tactile pressure stimulation and vibroacoustic vibration stimulation.

In some embodiments, the suspension framework comprises steel tubing, stainless steel, aluminum alloy, laminated hardwood, or combinations thereof.

In one embodiment, at least one rope of the plurality of ropes comprises hemp, jute, cotton, nylon, polyester, or blended fiber rope.

In one aspect, the vibroacoustic transducer is secured to the anchoring element by one or more fasteners comprising bolts, screws, or rivets.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of vibroacoustic rope-binding therapeutic system of the present invention in accordance with the disclosed structure;

FIG. 2 illustrates a functional block diagram showing internal components of the control unit of the vibroacoustic rope-binding therapeutic system of the present invention in accordance with the disclosed structure;

FIG. 3 illustrates a perspective view showing an exemplary therapeutic use configuration of the vibroacoustic rope-binding therapeutic system of the present invention in accordance with the disclosed structure;

FIG. 4 illustrates a flow diagram depicting steps of operating the vibroacoustic rope-binding therapeutic system to provide vibroacoustic stimulation in accordance with the disclosed structure; and

FIG. 5 illustrates an isolated close-up view of transducer-integrated suspension ring assembly of the vibroacoustic rope-binding therapeutic system of the present invention in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved therapeutic rope-binding system that overcomes limitations of conventional Shibari practices and conventional vibroacoustic therapy systems. There is a long-felt need for a specialized system that integrates Shibari-inspired decorative and grounding rope binding with controlled vibroacoustic stimulation in a safe, repeatable, and therapeutic manner. Additionally, there exists a need for an integrated device that delivers targeted low-frequency vibration through rope contact regions to enhance relaxation, reduce stress, and ease muscle tension while maintaining comfort and stability. Moreover, there is a need for a therapeutic system that enables customization of vibration parameters such as frequency, intensity, waveform, and modulation patterns to suit different users, sensitivities, and therapeutic objectives. Finally, there is a need for a durable, load-bearing, and user-friendly apparatus that can be deployed in clinical, wellness, or personal settings to expand access to these underutilized therapeutic practices.

The present invention, in one exemplary embodiment, is a method of providing vibroacoustic therapy through a rope-binding configuration and comprises generating, by a control unit, a therapeutic vibroacoustic drive signal and amplifying the therapeutic vibroacoustic drive signal to produce an amplified drive signal. The method further comprises transmitting the amplified drive signal from the control unit to a vibroacoustic transducer mechanically coupled to an anchoring element of a suspension ring assembly. The method further comprises converting, by the vibroacoustic transducer, the amplified drive signal into mechanical oscillation. The method further comprises vibrating the anchoring element and one or more ropes coupled thereto such that vibration propagates along tensioned rope pathways. The method further comprises delivering vibroacoustic stimulation to a user through one or more rope-body contact regions while the user is supported by the rope-binding configuration. As a result, the user experiences a combined multi-sensory therapeutic effect comprising tactile pressure stimulation and vibroacoustic vibration stimulation. The device may also be used by individuals who use hammocks. Further, it may be used for other industries, such as camping, lounging, yoga, aerial yoga, etc.

Reference will now be made in detail to the present preferred embodiments of the invention, examples of which are illustrated in the accompanying drawings. Wherever possible, the same reference numerals are used in the drawings and the description to refer to the same or like parts.

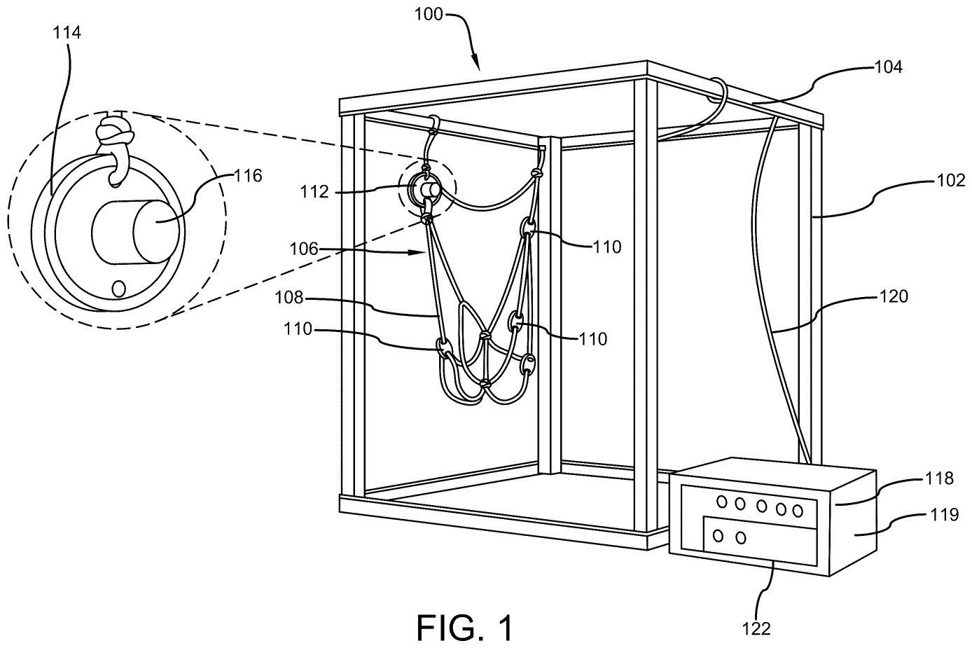

Referring initially to the drawings, FIG. 1 illustrates a perspective view of vibroacoustic rope-binding therapeutic system of the present invention in accordance with the disclosed structure. The vibroacoustic rope-binding therapeutic system 100 of the present invention is designed as a multi-sensory therapeutic system that combines Shibari-inspired rope binding and vibroacoustic therapy (VAT) through use of controlled, low-frequency vibration to provide relaxation, reduce stress, and ease muscle tension.

More specifically, the system 100 generally includes a suspension framework 102 that comprises a rigid, load-bearing structure. In the present embodiment, the framework 102 is shown as a freestanding four-post frame, however, in other embodiments, the framework 102 may be formed as a wall-mounted, ceiling-mounted, or truss-mounted structure. The suspension framework 102 is supported by an overhead support surface 104, which also helps in hanging ropes for rope-binding.

In some embodiments, the suspension framework 102 is formed of steel tubing, stainless steel, aluminum alloy, laminated hardwood, or combinations thereof. By way of non-limiting examples, structural members of the suspension framework 102 can have cross-sectional dimensions of approximately 25-75 mm (1-3 inches), and the overall framework (102) can have outer dimensions of approximately 1.5-3.0 m in height, 1.0-2.5 m in width, and 1.0-2.5 m in depth, to permit safe rope routing and suspension geometry. The suspension framework 102 is also secured to an overhead support surface 104 for Shibari-inspired rope binding.

The vibroacoustic rope-binding therapeutic system 100 further comprises a plurality of ropes 106 used to form at least one rope-binding configuration. Each rope 108 of the plurality of ropes 106 comprises an elongate flexible tensile member and is configured to create one or more tie points 110. The tie points 110 can include knots, bends, hitches, wraps, cinches, or other rope formations used to create geometric patterns and load distribution.

In some embodiments, the rope 108 comprises natural fiber rope such as hemp, jute, or cotton, or synthetic rope such as nylon, polyester, or blended fiber rope. In one embodiment, the rope 108 can have a diameter of about 4-12 mm, and preferably about 5-8 mm, to balance tactile comfort and vibration transmission. Each rope 108 can be treated, coated, or sleeved for grip characteristics and durability.

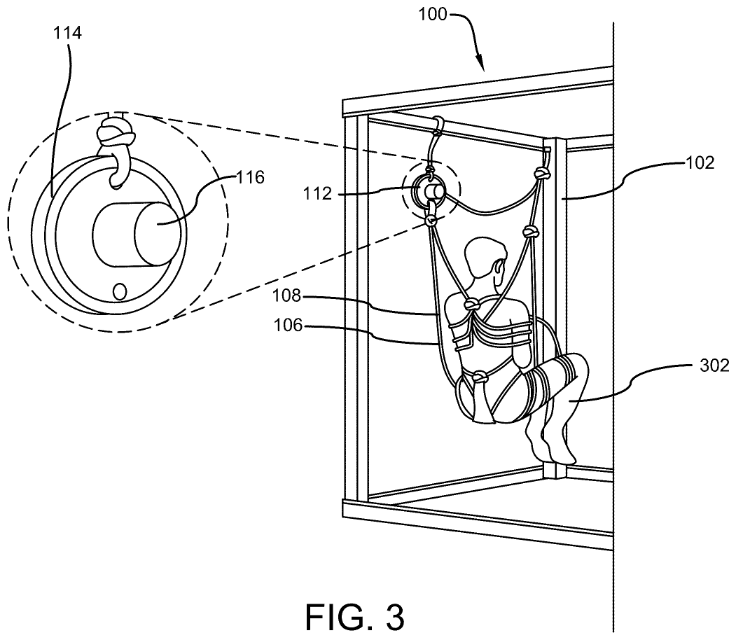

The plurality of ropes 106 is configured to receive vibroacoustic energy and transmit the energy along tensioned rope segments toward selected therapeutic contact regions when applied to a user (FIG. 3).

As shown, a suspension ring assembly 112 is coupled to the suspension framework 102 and receives at least one rope 108 of the plurality of ropes 106. It should be noted that the system 100 can include a plurality of suspension ring assemblies as per requirements of the users. The suspension ring assembly 112 comprises an anchoring element 114, which may be a closed-loop ring, D-ring, O-ring, carabiner-compatible ring, or equivalent mechanical coupling component configured to anchor and route rope 108. In some embodiments, the anchoring element 114 is formed from stainless steel, anodized aluminum, or other high-strength metals suitable for repeated load-bearing use.

The suspension ring assembly 112 further includes a vibroacoustic transducer 116 mechanically coupled to the anchoring element 114. In the present embodiment, the vibroacoustic transducer 116 is integrated with the ring assembly such that vibratory energy generated by the vibroacoustic transducer 116 is transferred into the anchoring element 114, and from the anchoring element 114 into the ropes 108 coupled thereto.

The vibroacoustic transducer 116 can comprise a tactile transducer, bass shaker, exciter, or equivalent electromechanical actuator operable to convert an electrical drive signal into mechanical oscillation. In some embodiments, the vibroacoustic transducer 116 is mounted within a cavity of the anchoring element 114, mounted to an outer surface using a mounting plate or fasteners, or bonded using an adhesive/epoxy coupling layer.

The vibroacoustic transducer 116 is configured to operate in a therapeutic low-frequency range of approximately 20-200 Hz and can be driven at power levels of approximately 10-200 W Root Mean Square (RMS) depending on the configuration and desired intensity.

The system 100 further includes a control unit 118 configured to control operation of the vibroacoustic transducer 116. The control unit 118 is coupled to the vibroacoustic transducer 116 via a wired connection 120, such as a shielded audio/power cable, multi-conductor cable, or dedicated actuator line. In some embodiments, the control unit 118 and the vibroacoustic transducer 116 are coupled via a wireless communication channel including but not limited to Bluetooth, NFC, or any other short-range communication channel.

The control unit 118 may be housed in a rigid enclosure 119 formed of metal, impact-resistant polymer, or composite materials. The control unit 118 can have dimensions about 150-450 mm wide, 100-300 mm high, and 150-400 mm deep, for portability and stable placement near the suspension framework 102.

The control unit 118 further includes a user interface 122 comprising one or more controls, such as knobs, dials, buttons, switches, indicator lights, and/or a display. The user interface 122 enables selection and adjustment of vibration parameters including at least frequency, amplitude/intensity, waveform shape, pattern modulation, duty cycle, and program duration.

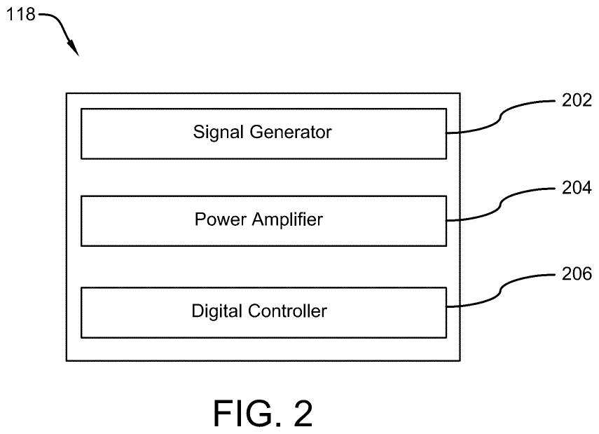

FIG. 2 illustrates a functional block diagram showing internal components of the control unit of the vibroacoustic rope-binding therapeutic system of the present invention in accordance with the disclosed structure. As shown, the control unit 118 comprises a plurality of functional modules including a signal generator 202, a power amplifier 204, and a digital controller 206, which work to generate, modulate, and deliver vibroacoustic drive signals to one or more vibroacoustic transducers 116.

The signal generator 202 is configured to produce one or more therapeutic vibroacoustic electrical drive signals. In some embodiments, the signal generator 202 generates signals depicting waveforms including, by way of non-limiting example, sinusoidal waves, square waves, triangle waves, sawtooth waves, noise-based signals, pulse trains, frequency sweeps, and preprogrammed pattern sequences. In one embodiment, the signal generator 202 preferably generates a vibroacoustic frequency range of about 20 Hz to 200 Hz and can generate in a broader vibroacoustic frequency range from 10 Hz to 500 Hz.

The signal generator 202 may be implemented using one or more analog oscillators, digital waveform synthesizers, a DSP digital signal processor, a microcontroller-generated waveform output, and/or stored audio playback.

The power amplifier 204 is configured to receive therapeutic vibroacoustic electrical drive signals from the signal generator 202 and provides amplified drive power to actuate the vibroacoustic transducer(s) 116. In some embodiments, the power amplifier 204 comprises an audio amplifier, a class D amplifier, or a class AB amplifier. The power amplifier 204 can provide output power in a range of approximately 10 W to 200 W RMS per channel and can provide a peak output capability exceeding RMS power to support transient therapeutic pulses.

The digital controller 206 is configured to control the operation of the signal generator 202 and power amplifier 204, and to implement selectable therapeutic program. The digital controller 206 may be implemented as a microcontroller, FPGA, DSP, or an embedded computer. The digital controller 206 is further configured to adjust output amplitude or frequency in response to detected load on ropes and thermal conditions.

In operation, the digital controller 206 selects vibroacoustic therapy parameters and commands the signal generator 202 to output a corresponding driving signal. The signal is then amplified by the power amplifier 204 to produce a transducer drive signal. The control unit 118 transmits the amplified drive signal through the wired connection 120, shown in FIG. 1, to actuate the vibroacoustic transducer 116, thereby imparting mechanical vibration to the anchoring element 114, suspension ring assembly 112, and rope system 106, as described in FIG. 1.

FIG. 3 illustrates a perspective view showing an exemplary therapeutic use configuration of the vibroacoustic rope-binding therapeutic system of the present invention in accordance with the disclosed structure. As shown, a user 302 is supported in a suspended seated posture by the suspension framework 102 through a plurality of ropes 106 arranged in a rope-binding configuration. The rope-binding configuration is formed to provide both structural support and controlled tactile contact with the user 302 at selected rope-body contact regions, thereby promoting grounding and relaxation.

In the present embodiment, at least one rope 108 of the plurality of ropes 106 is routed and tensioned to create a supportive harness around the user 302. The plurality of ropes 106 distribute load across multiple contact zones of the user 302, including one or more areas selected from the group consisting of the head, neck, torso, back, shoulders, arms, hips, buttocks, groin, legs, and feet to maintain comfort while providing secure restraint and a consistent tactile stimulus.

The plurality of ropes 106 are coupled to the one or more suspension ring assemblies 112, which serve as both a suspension anchor and a vibroacoustic stimulation node. The suspension ring assembly 112 includes the anchoring element 114 configured to engage and retain rope segments and to support dynamic loading during a therapy session. The vibroacoustic transducer 116 is mechanically integrated with the anchoring element 114 such that vibratory energy produced by the vibroacoustic transducer 116 is transmitted to the anchoring element 114 and into the attached rope segments.

During therapeutic operation, vibroacoustic stimulation generated by the vibroacoustic transducer 116 propagates along the tensioned rope pathways and into the user 302 through the rope-body contact regions. The vibroacoustic output may be applied as steady low-frequency vibration, pulsed patterns, rhythmic modulation, or frequency sweeps to achieve desired therapeutic outcomes. Accordingly, using the system 100, the user 302 experiences a combined multi-sensory therapeutic effect comprising tactile pressure stimulation from the rope-binding configuration and localized vibroacoustic vibration stimulation transmitted through the ropes. The system 100 provides benefits including stress reduction, muscle relaxation, stimulation, reduction of tension, and emotional regulation.

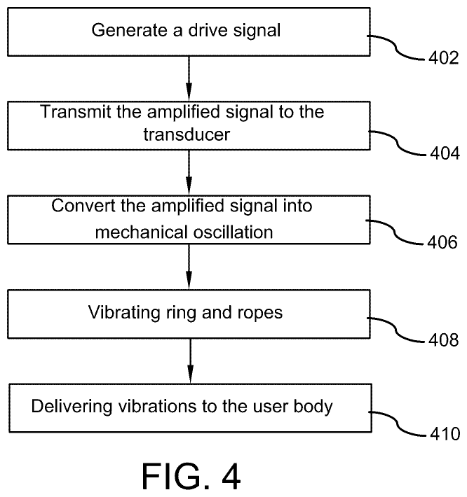

FIG. 4 illustrates a flow diagram depicting steps of operating the vibroacoustic rope-binding therapeutic system to provide vibroacoustic stimulation in accordance with the disclosed structure. Initially, the system 100 generates a drive signal (Step 402). Preferably, the drive signal is generated by the signal generator 202 under control of the digital controller 206 and can include a selected therapeutic program specifying one or more parameters comprising frequency, waveform shape, amplitude, modulation pattern, duty cycle, and session duration.

Then, the amplified signal is transmitted to the vibroacoustic transducer (Step 404). The drive signal is amplified by the power amplifier 204 and delivered from the control unit 118 to the vibroacoustic transducer 116 through the wired connection 120, which can include one or more conductors for power delivery and signal transmission.

Thereafter, the amplified signal is converted into mechanical oscillation (Step 406). The vibroacoustic transducer 116 converts the received electrical drive signal into physical vibration, thereby producing oscillatory mechanical movement corresponding to the therapeutic parameters.

Then, the suspension ring assembly and ropes are vibrated (Step 408). Mechanical oscillation from the vibroacoustic transducer 116 is mechanically coupled to the anchoring element 114 of the suspension ring assembly 112, causing the ring and connected rope segments of the plurality of ropes 106 to vibrate. The vibratory energy propagates along tensioned rope pathways and through tie points to maintain consistent vibrational transfer.

Finally, the vibrations are delivered to the user body (Step 410). The vibrating rope segments contact the user at one or more rope-body contact regions, thereby transmitting vibroacoustic stimulation into the user. Using the system 100, the method provides a combined therapeutic effect comprising tactile pressure stimulation and vibration stimulation to promote relaxation, reduce stress, relieve muscle tension, and support emotional regulation.

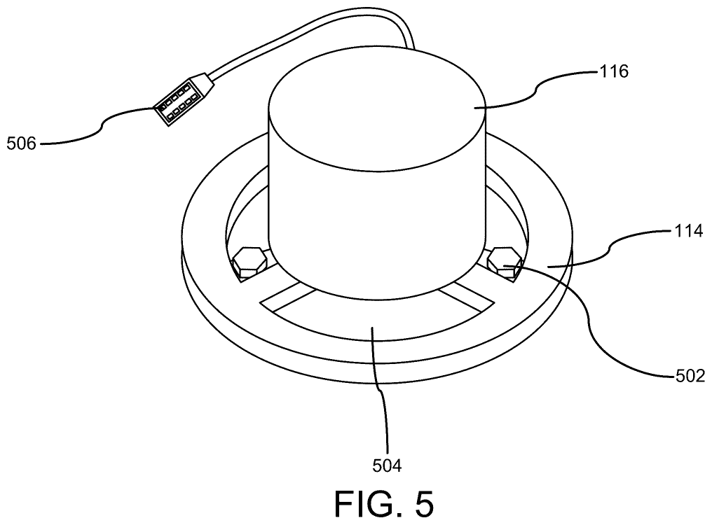

FIG. 5 illustrates an isolated close-up view of transducer-integrated suspension ring assembly of the vibroacoustic rope-binding therapeutic system of the present invention in accordance with the disclosed structure. As shown, the assembly 112 includes the anchoring element 114 configured as a closed-loop ring component for receiving and retaining one or more ropes of the rope system. The anchoring element 114 is formed as a rigid ring having a generally circular geometry and defining a central opening through which the vibroacoustic transducer is mounted.

The vibroacoustic transducer 116 is mechanically coupled to the anchoring element 114 and is configured to convert an electrical drive signal into mechanical oscillation. The vibroacoustic transducer 116 is secured to the anchoring element 114 by one or more fasteners 502, such as bolts, screws, or rivets, which mechanically clamp the vibroacoustic transducer 116 to the anchoring element 114. In some embodiments, an intermediate coupling interface (not shown), such as a mounting plate, gasket, vibration coupling layer, or adhesive/epoxy layer, may be positioned between the vibroacoustic transducer 116 and the anchoring element 114 to improve vibration transfer and reduce relative movement during operation.

The anchoring element 114 further includes a rope-routing region 504 defined along a peripheral portion of the ring, which provides a contact surface for rope engagement and load transfer.

The vibroacoustic transducer 116 is electrically connected to the control unit by a wired connection 506, such as a shielded cable comprising one or more conductors for power delivery and signal transmission. In operation, the wired connection 506 transmits amplified drive signals from the control unit to the vibroacoustic transducer 116, causing the vibroacoustic transducer 116 to vibrate.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “vibroacoustic rope-binding therapeutic system”, “rope-binding therapeutic system”, and “system” are interchangeable and refer to the vibroacoustic rope-binding therapeutic system 100 of the present invention.

Notwithstanding the forgoing, the vibroacoustic rope-binding therapeutic system 100 of the present invention can be of any suitable configuration as is known in the art without affecting the overall concept of the invention, provided that it accomplishes the above stated objectives. One of ordinary skill in the art will appreciate that the vibroacoustic rope-binding therapeutic system 100 shown in the FIGS. are for illustrative purposes only, and that many other configurations of the vibroacoustic rope-binding therapeutic system 100 are well within the scope of the present disclosure. Although the dimensions of the vibroacoustic rope-binding therapeutic system 100 are important design parameters for user convenience, the vibroacoustic rope-binding therapeutic system 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A vibroacoustic rope-binding therapeutic system comprising:
a suspension framework;
a plurality of ropes;
anchoring elements;
vibroacoustic energy;
therapeutic contact regions;
a suspension ring assembly; and
a vibroacoustic transducer;
wherein said suspension framework comprises a rigid load-bearing structure;
wherein said suspension framework selected from the group consisting of a freestanding four-post frame, a wall-mounted structure, a ceiling-mounted structure, and a truss-mounted structure;
wherein said suspension framework is supported by an overhead support surface for hanging said plurality of ropes and said anchoring elements;
wherein said plurality of ropes are configured to form at least one rope-binding configuration;
wherein each rope of said plurality of ropes comprises an elongate flexible tensile member configured to create one or more tie points;
wherein said tie points comprise connections selected from the group consisting of knots, bends, hitches, wraps, and cinches to create geometric patterns and load distribution;
wherein said plurality of ropes comprise a diameter from 4 mm to 12 mm for tactile comfort and vibration transmission;
wherein said plurality of ropes is configured to receive said vibroacoustic energy and transmit said vibroacoustic energy along tensioned rope segments toward selected said therapeutic contact regions when applied to a user;
wherein said suspension ring assembly is coupled to said suspension framework and receives at least one rope of said plurality of ropes; and
further wherein said vibroacoustic transducer is integrated with said suspension ring assembly and configured for transferring said vibroacoustic energy generated by said vibroacoustic transducer into said anchoring elements and from said anchoring elements to said plurality of ropes.

2. The vibroacoustic rope-binding therapeutic system of claim 1, wherein said vibroacoustic transducer comprises one or more selected from the group consisting of a tactile transducer, a bass shaker, an exciter, and an electromechanical actuator operable to convert an electrical drive signal into mechanical oscillation.

3. The vibroacoustic rope-binding therapeutic system of claim 1, wherein said vibroacoustic transducer is configured to operate in a therapeutic low-frequency range of approximately 20 Hz to 200 Hz.

4. The vibroacoustic rope-binding therapeutic system of claim 1, wherein said vibroacoustic transducer is configured to be driven at power levels from 10 W to 200 W Root Mean Square (RMS).

5. The vibroacoustic rope-binding therapeutic system of claim 1, wherein said suspension ring assembly comprises a plurality of anchoring elements selected from the group consisting of a closed-loop ring, a D-ring, an O-ring, and a carabiner-compatible ring configured to anchor and route said plurality of ropes.

6. The vibroacoustic rope-binding therapeutic system of claim 1, wherein said plurality of ropes comprise natural fiber rope selected from the group consisting of hemp, jute, and cotton.

7. The vibroacoustic rope-binding therapeutic system of claim 1, wherein said plurality of ropes comprises synthetic rope selected from the group consisting of nylon, polyester, and blended fiber rope.

8. The vibroacoustic rope-binding therapeutic system of claim 1 further comprising a control unit configured to control operation of said vibroacoustic transducer, wherein said control unit is coupled to said vibroacoustic transducer and comprises a user interface configured to adjust vibration parameters selected from the group consisting of frequency, amplitude, intensity, waveform shape, pattern modulation, duty cycle, and program duration.

9. The vibroacoustic rope-binding therapeutic system of claim 8, wherein said vibration parameters selected from the group consisting of sinusoidal waves, square waves, triangle waves, sawtooth waves, noise-based signals, pulse trains, frequency sweeps, and preprogrammed pattern sequences.

10. The vibroacoustic rope-binding therapeutic system of claim 9, wherein said control unit comprises a signal generator configured to generate a vibroacoustic frequency range from 10 Hz to 500 Hz.

11. The vibroacoustic rope-binding therapeutic system of claim 10, wherein said signal generator selected from the group consisting of an analog oscillator, a digital waveform synthesizers, a DSP digital signal processor, and a microcontroller-generated waveform output.

12. The vibroacoustic rope-binding therapeutic system of claim 11, wherein said control unit comprises a power amplifier configured to receive therapeutic vibroacoustic electrical drive signals from said signal generator and provides amplified drive power to actuate said vibroacoustic transducer, wherein said power amplifier selected from the group consisting of an audio amplifier, a class D amplifier, and a class AB amplifier.

13. The vibroacoustic rope-binding therapeutic system of claim 12, wherein said control unit transmits said therapeutic vibroacoustic electrical drive signals to impart mechanical vibration to said anchoring element, said suspension ring assembly, and said plurality of ropes.

14. The vibroacoustic rope-binding therapeutic system of claim 1, wherein said rope-binding configuration is routed and tensioned to form a suspension harness for distributing loads across multiple said therapeutic contact regions of the user, wherein said therapeutic contact regions selected from one or more of the group consisting of a head, a neck, a torso, a back, shoulders, arms, hips, buttocks, groin, legs, and feet.

15. A vibroacoustic rope-binding therapeutic system comprising:
a suspension framework;
a plurality of ropes;
anchoring elements;
vibroacoustic energy;
therapeutic contact regions;
a suspension ring assembly;
a vibroacoustic transducer; and
a control unit;
wherein said suspension framework comprises a rigid load-bearing structure;
wherein said suspension framework selected from the group consisting of a freestanding four-post frame, a wall-mounted structure, a ceiling-mounted structure, and a truss-mounted structure;
wherein said suspension framework is supported by an overhead support surface for hanging said plurality of ropes and said anchoring elements;
wherein said plurality of ropes are configured to form at least one rope-binding configuration;
wherein said plurality of ropes configured to create one or more tie points;
wherein said tie points comprise connections selected from the group consisting of knots, bends, hitches, wraps, and cinches to create geometric patterns and load distribution;
wherein said plurality of ropes is configured to receive said vibroacoustic energy and transmit said vibroacoustic energy along tensioned rope segments toward selected said therapeutic contact regions when applied to a user;
wherein said suspension ring assembly is coupled to said suspension framework and receives at least one rope of said plurality of ropes;
wherein said vibroacoustic transducer is integrated with said suspension ring assembly and configured for transferring said vibroacoustic energy generated by said vibroacoustic transducer into said anchoring elements and from said anchoring elements to said plurality of ropes;
wherein said rope-binding configuration is routed and tensioned to form a suspension harness for distributing loads across multiple said therapeutic contact regions of the user, wherein said therapeutic contact regions selected from one or more of the group consisting of a head, a neck, a torso, a back, shoulders, arms, hips, buttocks, groin, legs, and feet; and
further wherein said control unit coupled to said vibroacoustic transducer and comprises a user interface configured to adjust vibration parameters selected from the group consisting of frequency, amplitude, intensity, waveform shape, pattern modulation, duty cycle, and program duration.

16. The vibroacoustic rope-binding therapeutic system of claim 15, wherein said vibration parameters selected from the group consisting of sinusoidal waves, square waves, triangle waves, sawtooth waves, noise-based signals, pulse trains, frequency sweeps, and preprogrammed pattern sequences.

17. The vibroacoustic rope-binding therapeutic system of claim 15, wherein said control unit comprises a power amplifier configured to receive therapeutic vibroacoustic electrical drive signals from a signal generator and provides amplified drive power to actuate said vibroacoustic transducer, wherein said power amplifier selected from the group consisting of an audio amplifier, a class D amplifier, and a class AB amplifier.

18. The vibroacoustic rope-binding therapeutic system of claim 17, wherein said control unit transmits said therapeutic vibroacoustic electrical drive signals to impart mechanical vibration to said anchoring elements, said suspension ring assembly, and said plurality of ropes.

19. A method of vibroacoustic rope-binding therapy, the method comprising the steps of:
providing a suspension framework, a plurality of ropes, anchoring elements, vibroacoustic energy, therapeutic contact regions, a suspension ring assembly, and a vibroacoustic transducer;
wherein said suspension framework selected from the group consisting of a freestanding four-post frame, a wall-mounted structure, a ceiling-mounted structure, and a truss-mounted structure;
supporting an overhead support surface with said suspension framework for hanging said plurality of ropes and said anchoring elements;
wherein said plurality of ropes are configured to form at least one rope-binding configuration;
creating one or more tie points with said plurality of ropes, wherein said tie points comprise connections selected from the group consisting of knots, bends, hitches, wraps, and cinches to create geometric patterns and load distribution;
wherein said plurality of ropes is configured to receive said vibroacoustic energy;
transmitting said vibroacoustic energy along tensioned rope segments toward selected said therapeutic contact regions when applied to a user;
coupling said suspension ring assembly to said suspension framework for receiving at least one rope of said plurality of ropes;
integrating said vibroacoustic transducer with said suspension ring assembly;
transferring said vibroacoustic energy generated by said vibroacoustic transducer into said anchoring elements and from said anchoring elements to said plurality of ropes;
wherein said rope-binding configuration is routed and tensioned to form a suspension harness for distributing loads across multiple said therapeutic contact regions of the user; and
wherein said therapeutic contact regions selected from one or more of the group consisting of a head, a neck, a torso, a back, shoulders, arms, hips, buttocks, groin, legs, and feet.

20. The method of vibroacoustic rope-binding therapy of claim 19 further comprising a control unit, wherein said control unit coupled to said vibroacoustic transducer and comprises a user interface configured to adjust vibration parameters selected from the group consisting of frequency, amplitude, intensity, waveform shape, pattern modulation, duty cycle, and program duration.
